(19) United States
(12) Patent Application Publication
(10) Pub. No.: US 2016/0266125 A1
(43) Pub. Date: Sep. 15, 2016

(54) METHODS OF TREATING AND PREDICTING PROGRESSION OF CANCER BASED ON T CELL SUBSETS

(71) Applicant: JOHN WAYNE CANCER INSTITUTE, Santa Monica, CA (US)

(72) Inventors: Peter Allan Sieling, Pacific Palisades, CA (US); Marilou Terpenning, Pacific Palisades, CA (US); Delphine J. Lee, Los Angeles, CA (US)

(21) Appl. No.: 15/069,779

(22) Filed: Mar. 14, 2016

Related U.S. Application Data
(60) Provisional application No. 62/132,500, filed on Mar. 12, 2015.

(51) Int. Cl. G01N 33/574

(52) U.S. Cl. CPC G01N 33/57415; G01N 2800/56; G01N 2800/7028

(57) ABSTRACT

Disclosed are methods of predicting whether a subject's metastatic breast cancer is stable or progressive are provided. Such methods may include measuring T cell subsets in a fluid sample of the subject; and predicting whether the subject's metastatic breast cancer is likely to be stable or progressive when there is an elevated ratio of the T cell subsets. Also disclosed are methods of treating metastatic breast cancer by a conservative therapy or an aggressive therapy based on the determination of a stable or progressive cancer.

### CROSS-REFERENCE TO RELATED APPLICATIONS

This application claims the benefit of U.S. Provisional Patent Application No. 62/132,500, filed Mar. 12, 2015 and now pending, the content of which is incorporated herein by reference in its entirety.

### BACKGROUND

Approximately 232,670 new cases of invasive breast cancer were expected to be diagnosed in women in the U.S. in 2014, and approximately 40,000 women in the U.S. were expected to die in 2014 from breast cancer. Death from breast cancer occurs as a result of metastatic disease. Although the incidence of breast cancer death has declined due to increased screening and improved local and systemic therapies, subpopulations of women with breast cancer still recur after initial therapy.

T cells are white blood cells that mediate and regulate immunity against microbial infection and cancer. Control of breast cancer progression may be mediated by the host antitumor response (Mahmoud et al., 2011); however, the specific molecules responsible for antitumor immunity in breast cancer are not fully defined (Oldford et al., 2006; Teschendorff et al., 2010; Camp et al., 1996; Bates et al., 2006; Mahmoud et al., 2011; Yoon et al., 2010). A robust role played by the immune system in control of breast cancer is suggested by studies showing the presence of T cells (Ali et al., 2014), particularly CD8 T cells, predicts clinical outcome in breast cancer (Mahmoud, 2011), though the role of T cell subsets and their mediators is unclear (La Rocca et al., 2008; Sheu et al., 2008; Aaltomaa et al., 1992; Matkowski et al., 2009).

The success of cancer immunotherapies (Hodi et al., 2010; Topalian et al., 2012; Brahmer et al., 2012) has transformed cancer treatment and confirmed the role of the immune system in fighting cancer. However, conflicting reports indicate that the immune system, particularly T cells, can inhibit or promote the growth of tumors (Fridman et al., 2012). This dichotomous relationship between the immune system and cancer can be explained by the concept of cancer immune editing (Vesely et al., 2013).

This is a dynamic process, whereby the immune system not only protects against cancer development but selects for tumors that suppress or evade the immune system to persist. Cancer immune editing may explain why the role of T cells in breast cancer is not entirely clear, because although increased numbers of tumor infiltrating T cells have been reported to be associated with good clinical outcome in some studies (Mahmoud et al., 2011; La Rocca et al., 2008), the opposite has been noted by others (Sheu et al., 2008; Aaltomaa et al., 1992; Rafal et al., 2009).

T lymphocytes inhibit tumor growth by i) direct lysis of tumor via release of perforin and granzymes; ii) secretion of soluble mediators, including cytokines and chemokines that recruit host cells to lyse tumor, and iii) licensing antigen presenting cells to activate cytolytic CD8 T cells. A dysregulated immune response can in contrast, promote tumor growth by inducing a wound healing response that includes recruitment and differentiation of macrophages and T cells that suppress cytotoxic activities of CD8 and Th1 cells. Therefore, defining the molecular characteristics of T cells in patients with good vs. poor outcome becomes very important in improving treatment of metastatic breast cancer.

Metastatic breast cancer can remain stable for years during treatment, while some patients' disease progresses and they die. Determining whether a breast cancer patient's metastatic disease will progress is an important factor in determining the choice of aggressive or conservative treatment. It would therefore be beneficial to predict progressive metastatic breast cancer.

### BRIEF DESCRIPTION OF THE DRAWINGS

FIG. 1 shows that the ratio of regulatory T cells (Treg) to CD4 T cells was elevated in three patients with progressive metastatic disease, relative to four patients with stable metastatic disease.

### DETAILED DESCRIPTION

One aspect of the disclosure relates to a method for predicting in a subject having metastatic breast cancer whether the metastatic breast cancer is stable or progressive, the method comprising:
measuring two or more T cell subsets in a fluid sample obtained from the subject;
calculating the ratio of the T cell subsets; and
predicting whether the metastatic breast cancer is stable or progressive, according to the ratio of the T cell subsets.

In a related aspect, the disclosure relates to a method for treating a subject having a stable metastatic breast cancer or a progressive metastatic breast cancer, the method comprising:
measuring two or more T cell subsets in a fluid sample obtained from the subject;
calculating the ratio of the T cell subsets;
predicting whether the metastatic breast cancer is stable or progressive, according to the ratio of the T cell subsets; and
administering a conservative therapy to the subject having a stable metastatic breast cancer, or administering an aggressive therapy to the subject having a progressive metastatic breast cancer.

Conservative and aggressive therapies for metastatic breast cancer are known in the art. For example, some aggressive therapies include surgery, radiation therapy, chemotherapy, and a combination thereof. Some examples of conservative therapies include an endocrine therapy (or an anti-estrogen treatment), a biologic therapy that targets a specific protein or pathway. It is within the purview of one of ordinary skill in the art to select one or more suitable therapy based on the status of the metastatic breast cancer. Additional, one would understand that some conservative therapies and aggressive therapies may overlap with each other.

In certain embodiments, the breast cancer is ER+ breast cancer Stage IIB or higher, triple negative breast cancer (TNBC) Stage I or higher, Her2+ breast cancer Stage IIA or higher.

Examples of the fluid sample include, without limitation, peripheral blood, plasma, serum, immune cells, and any other fluid found in the body that may contain T cell subsets.

In certain embodiments, the fluid sample is peripheral blood. Although the tumor and surrounding microenvironment provides insight into the mechanisms of disease progression, the peripheral blood frequently contains physiological remnants of these mechanisms; moreover, peripheral blood is more readily sampled for prognostic markers than tumor tissue. As described herein, the systemic immune compartment reflected in peripheral blood may regulate spread of disease to distant sites. For example, the Treg/CD4 T cells ratio may be associated with systemic metastases.

Examples of the T cell subsets include, without limitation, CD8 cytotoxic T cells (CTL), T helper 1 (Th1) cells, Th2 cells, Th1/17 cells, Th17 cells, and Treg. In certain embodiments, T cell subsets include the Treg cells and CD4 T cells.

In certain embodiments, the ratio of the T cell subsets is Treg/CD4 T cells; and the subject's breast cancer is more likely to be progressive metastatic breast cancer if the ratio of the T cell subsets is greater than about 0.30, greater than about 0.29, greater than about 0.28, greater than about 0.27, greater than about 0.26, greater than about 0.25, greater than about 0.24, greater than about 0.23, greater than about 0.22, or greater than about 0.21, greater than about 0.20, greater than about 0.19, greater than about 0.18, greater than about 0.17, greater than about 0.16, greater than about 0.15, greater than about 0.14, greater than about 0.13, greater than about 0.12, or greater than about 0.11. Unexpectedly, the Treg/CD4 T cells ratios in patients with progressive metastatic disease have been found to be significantly higher than those in patients with stable metastatic disease. As used herein, the term “about” means a range of ±10%, ±5%, or ±1%.

Cell surface markers may be identified by flow cytometry. Soluble mediators may be identified by culturing peripheral blood T cells with autologous tumor (if available), autologous monocyte-derived dendritic cells pulsed with tumor antigen, or anti-CD3/CD28 beads, and response may be measured by ELISPOT or Luminex. Soluble factors may be identified in serum as well by Luminex.

In some embodiments, the methods for measuring the ratio of regulatory T cells (Treg) to CD4 T cells in peripheral blood may include steps of (1) labeling cells from the peripheral blood with fluorescent antibodies that detect Treg, including CD3, CD4, CD25, and CD127 as well as a dye that discriminates live from dead cells, (2) acquiring the labeled cells in a flow cytometer, which detects the fluorescence levels as an indicator of protein expression; (3) determining the frequency of Treg cells (CD3+, CD4+, CD25+, CD127−, live) and the frequency of CD4 T cells (CD3+, CD4+, live) by analysis software; (4) calculating the ratio of Treg to CD4 T cells. The examples described herein indicate that elevated ratios of Treg to CD4 T cells are predictive of progressive metastatic breast cancer.

The following examples are intended to illustrate various embodiments of the disclosure. As such, the specific embodiments discussed are not to be construed as limitations on the scope of the disclosure. It will be apparent to one skilled in the art that various equivalents, changes, and modifications may be made without departing from the scope of disclosure, and it is understood that such equivalent embodiments are to be included herein. Further, all references cited in the disclosure are hereby incorporated by reference in their entirety, as if fully set forth herein.

### EXAMPLES

### Example 1

### Peripheral blood T cell subsets associated with progressive disease compared with stable disease in women receiving treatment for metastatic breast cancer

As described herein, certain peripheral blood T cell subsets were shown to be associated with progressive metastatic breast cancer compared with stable metastatic breast cancer in women receiving treatment for metastatic breast cancer.

Breast cancer patients identified as high risk for recurrence were identified by a medical oncologist and recruited to the JWCI biospecimen repository. Patients at high risk for metastatic disease include, women with ER+ breast cancer Stage IIB or higher at diagnosis; women with triple negative breast cancer (TNBC) Stage I or higher; and women with Her2+ breast cancer Stage IIA or higher. Blood was drawn, and peripheral blood mononuclear cells (PBMC) were isolated by density gradient centrifugation of blood in anticoagulant; serum was isolated from anticoagulant free tubes. PBMC were immediately labeled for phenotyping or frozen. Serum was frozen in aliquots to prevent the necessity of multiple freeze-thaw rounds. An IRB-exempt protocol allowed for clinical information along with the tissue and blood specimens to be utilized.

Cell surface markers were identified by flow cytometry. Soluble mediators were identified by culturing peripheral blood T cells with autologous tumor, autologous monocyte-derived dendritic cells pulsed with tumor antigen, or anti-CD3/CD28 beads, and response measured by ELISPOT or Luminex. Soluble factors were identified in serum as well by Luminex.

Multiparameter flow cytometry. To identify T cells and broad populations, cells were surface labeled with anti-human CD3, CD4, and CD8 Abs. T cell subsets, including Treg (Liu, 2006), Th1, Th2, Th17, and nonconventional Th1 (defined as Th1*) that produce IFN- and low levels of IL-17 (Acosta-Rodriguez, 2007) were measured. Memory and effector T cell subsets including T_{CM}, T_{EM}, T_{RM}, effector (T_{EFF}), T effector memory RA (T_{EMRA}). CTLA-4 and TIM3 expression were also measured as T cell dysfunction markers. Flow cytometry acquisition was conducted using LSRII (BD Biosciences), and the data analyzed using FlowJo software (Tree Star, Inc.).

In one embodiment, blood was drawn from patients having metastatic breast cancer. Cells were isolated for measurement of the ratio of Treg to CD4 T cells according to the protocol described below.

Flow Labeling For Regulatory T Cells (Treg)

I) Live/Dead (Fixable Viability Dye (FVD) eFluor 780). The vial of Fixable Viability Dye (FVD) was allowed to equilibrate to room temperature before opening.

Labeling with Fixable Viability Dye was done in azide-free and serum/protein-free PBS. Labeling in less than 0.5 mL was not recommended. The cells were prepared as desired in tubes. Only the tubes that needed Live/Dead labeling were taken, and the tubes that did not need live/dead labeling were set aside. The cells were washed 2 times in azide-free and serum/protein-free PBS, and then were resuspended at 1-10×10⁶/mL in azide-free and serum/protein-free PBS. 1 μL of Fixable Viability Dye per 1 mL of cells was added and vortexed immediately. The cells were incubated for 30 min at 2-8° C., protect from light. Subsequently, the cells were washed 1-2 times with flow labeling buffer (FACS buffer) or equivalent.

II) Cell Surface labeling. Tubes with unlabeled cells were set aside. A master mix of FACS buffer+50% AB Human Serum was made and 100 μL of this was added to each tube. 5 μl of each of the following antibodies was added to each tube: CD4 FITC, CD25 PE, CD127 PerCP-Cy5.5, and CD3 PE Cy7. The tubes were incubated for 20 minutes in the dark and then washed with FACS buffer. The labeled cells were resuspended in an appropriate volume of Flow Cytometry Labeling Buffer and samples were acquired on a flow cytometer.

III) Data analysis. FlowJo software was used to determine the frequency of Treg cells in a specimen. Instructions were set to exclude dead cells, gate on CD3+, gate on single cells, then CD4+. Then, a dot or contour plot was created showing CD25 and CD127 expression. The frequency of CD25+, CD127− cells in the CD4 population was quantified. A ratio of Treg to CD4 T cells was calculated.

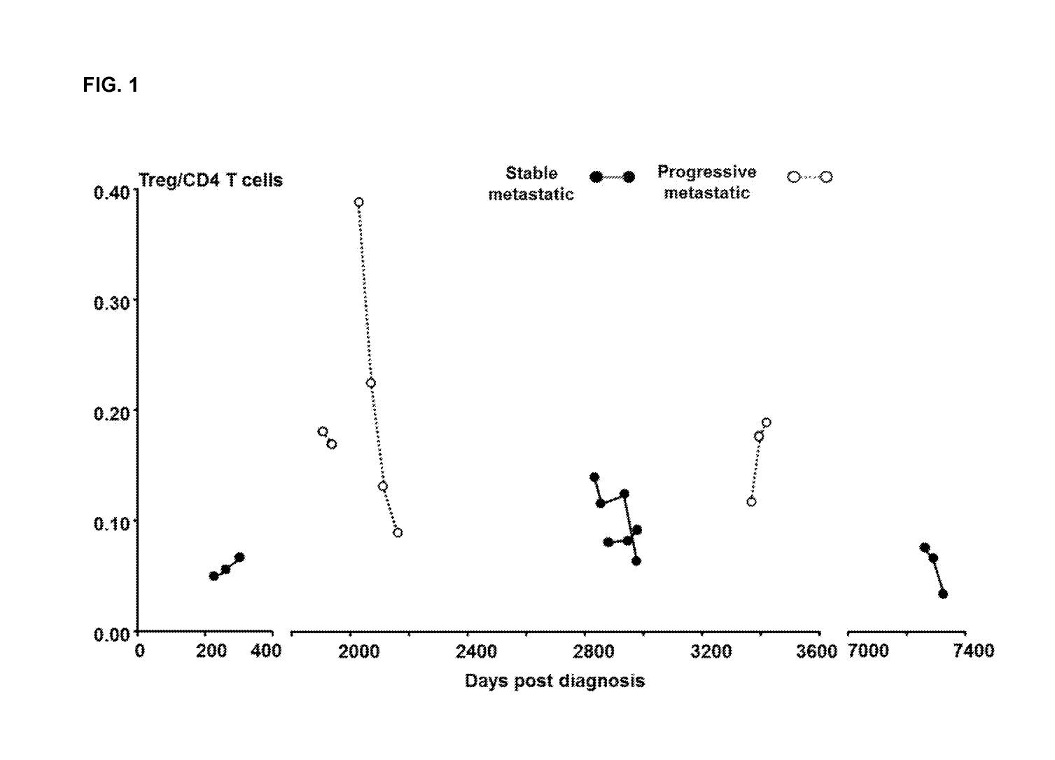

It was found that the ratio of Treg to CD4 T cells was elevated in three patients with progressive metastatic disease, relative to four patients with stable metastatic disease over time (FIG. 1). The ratios of Treg to CD4 T cells in progressive metastatic disease were mostly greater than 0.15.

### REFERENCES

The references, patents and published patent applications listed below, and all references cited in the specification above are hereby incorporated by reference in their entirety, as if fully set forth herein.
1. Zou W, Restifo N P. Nine lives for TH9s? Nat Med. 2012; 18(8):1177-8. doi: 10.1038/nm.2868. PubMed PMID: 22869182.
2. Mahmoud S M A, Paish E C, Powe D G, Macmillan R D, Grainge M J, Lee A H S, Ellis I O, Green A R. Tumor-Infiltrating CD8+ Lymphocytes Predict Clinical Outcome in Breast Cancer. Journal of Clinical Oncology. 2011; 29(15):1949-55. doi: 10.1200/jco.2010.30.5037.
3. Oldford S A, Robb J D, Codner D, Gadag V, Watson P H, Drover S. Tumor cell expression of HLA-DM associates with a Th1 profile and predicts improved survival in breast carcinoma patients. International Immunology. 2006; 18(11):1591-602. doi: 10.1093/intimm/dx1092.
4. Teschendorff A, Gomez S, Arenas A, El-Ashry D, Schmidt M, Gehrmann M, Caldas C. Improved prognostic classification of breast cancer defined by antagonistic activation patterns of immune response pathway modules. BMC Cancer. 2010; 10(1):604. PubMed PMID: doi: 10.1186/1471-2407-10-604.
5. Camp B J, Dyhrman S T, Memoli V A, Mott L A, Barth R J, Jr. In situ cytokine production by breast cancer tumor-infiltrating lymphocytes. Ann Surg Oncol. 1996; 3(2):176-84. PubMed PMID: 8646519.
6. Bates G J, Fox S B, Han C, Leek R D, Garcia J F, Harris A L, Banham A H. Quantification of Regulatory T Cells Enables the Identification of High-Risk Breast Cancer Patients and Those at Risk of Late Relapse. Journal of Clinical Oncology. 2006; 24(34):5373-80. doi: 10.1200/jco.2006.05.9584.
7. Mahmoud S M, Paish E C, Powe D G, Macmillan R D, Lee A H, Ellis I O, Green A R. An evaluation of the clinical significance of FOXP3+ infiltrating cells in human breast cancer. Breast Cancer Res Treat. 2011; 127(1):99-108. doi: 10.1007/s10549-010-0987-8. PubMed PMID: 20556505.
8. Yoon N K, Maresh E L, Shen D, Elshimali Y, Apple S, Horvath S, Mah V, Bose S, Chia D, Chang H R, Goodglick L. Higher levels of GATA3 predict better survival in women with breast cancer. Human pathology. 2010; 41(12):1794-801. doi: 10.1016/j.humpath.2010.06.010. PubMed PMID: 21078439; PubMed Central PMCID: PMC2983489.
9. Ali H R, Provenzano E, Dawson S-J, Blows F M, Liu B, Shah M, Earl H M, Poole C J, Hiller L, Dunn J A, Bowden S J, Twelves C, Bartlett J M S, Mahmoud S M A, Rakha E, Ellis I O, Liu S, Gao D, Nielsen T O, Pharoah P D P, Caldas C. Association between CD8+ T-cell infiltration and breast cancer survival in 12 439 patients. Annals of Oncology. 2014; 25(8):1536-43. doi: 10.1093/annonc/mdu191.
10. La Rocca G, Anzalone R, Corrao S, Magno F, Rappa F, Marasà S, Czarnecka A M, Marasà L, Sergi C, Zummo G, Cappello F. CD1a down-regulation in primary invasive ductal breast carcinoma may predict regional lymph node invasion and patient outcome. Histopathology. 2008; 52(2):203-12. doi: 10.1111/j.1365-2559.2007.02919.x.
11. Sheu B-C, Kuo W-H, Chen R-J, Huang S-C, Chang K-J, Chow S-N. Clinical significance of tumor-infiltrating lymphocytes in neoplastic progression and lymph node metastasis of human breast cancer. The Breast. 2008; 17(6):604-10. doi: 10.1016/j.breast.2008.06.001.
12. Aaltomaa S, Lipponen P, Eskelinen M, Kosma V M, Marin S, Alhava E, Syrjanen K. Lymphocyte infiltrates as a prognostic variable in female breast cancer. European journal of cancer. 1992; 28A(4-5):859-64. PubMed PMID: 1524909.
13. Matkowski R, Gisterek I, Halon A, Lacko A, Szewczyk K, Staszek U, Pudelko M, Szynglarewicz B, Szelachowska J, Zolnierek A, Kornafel J. The prognostic role of tumor-infiltrating CD4 and CD8 T lymphocytes in breast cancer. Anticancer Res. 2009; 29(7):2445-51. PubMed PMID: 19596912.
14. Hodi F S, O'Day S J, McDermott D F, Weber R W, Sosman J A, Haanen J B, Gonzalez R, Robert C, Schadendorf D, Hassel J C, Akerley W, van den Eertwegh A J M, Lutzky J, Lorigan P, Vaubel J M, Linette G P, Hogg D, Ottensmeier C H, Lebbe C, Peschel C, Quirt I, Clark J I, Wolchok J D, Weber J S, Tian J, Yellin M J, Nichol G M, Hoos A, Urba W J. Improved Survival with Ipilimumab in Patients with Metastatic Melanoma. New England Journal of Medicine. 2010; 363(8):711-23. doi: doi:10.1056/NEJMoa1003466. PubMed PMID: 20525992.
15. Topalian S L, Hodi F S, Brahmer J R, Gettinger S N, Smith D C, McDermott D F, Powderly J D, Carvajal R D, Sosman J A, Atkins M B, Leming P D, Spigel D R, Antonia S J, Horn L, Drake C G, Pardoll D M, Chen L, Sharfman W H, Anders R A, Taube J M, McMiller T L, Xu H, Korman A J, Jure-Kunkel M, Agrawal S, McDonald D, Kollia G D, Gupta A, Wigginton J M, Sznol M. Safety, Activity, and Immune Correlates of Anti-PD-1 Antibody in Cancer. New England Journal of Medicine. 2012; 366(26):2443-54. doi: doi:10.1056/NEJMoa1200690. PubMed PM ID: 22658127.
16. Brahmer J R, Tykodi S S, Chow L Q M, Hwu W-J, Topalian S L, Hwu P, Drake C G, Camacho L H, Kauh J, Odunsi K, Pitot H C, Hamid O, Bhatia S, Martins R, Eaton K, Chen S, Salay T M, Alaparthy S, Grosso J F, Korman A J, Parker S M, Agrawal S, Goldberg S M, Pardoll D M, Gupta A, Wigginton J M. Safety and Activity of Anti-PD-L1 Antibody in Patients with Advanced Cancer. New England Journal of Medicine. 2012; 366(26):2455-65. doi: doi:10.1056/NEJMoa1200694. PubMed PMID: 22658128.
17. Fridman W H, Pagès F, Sautès-Fridman C, Galon J. The immune contexture in human tumours: impact on clinical outcome. Nat Rev Cancer. 2012; 12(4):298-306.
18. Vesely M D, Schreiber R D. Cancer immunoediting: antigens, mechanisms, and implications to cancer immunotherapy. Annals of the New York Academy of Sciences. 2013; 1284(1):1-5. doi: 10.1111/nyas.12105.
19. Rafal M, Gisterik I, Halon A, Lacko A, Szewczyk K, Staszek U, Pudelko M, Szynglarewicz B, Szelachowska J, Zolnierek A, Kornafel J. The Prognostic Role of Tumor-infiltrating CD4 and CD8 T Lymphocytes in Breast Cancer. Anticancer Research. 2009; 29(7):2445-51.
20. Clark R A, Chong B F, Mirchandani N, Yamanaka K-I, Murphy G F, Dowgiert R K, Kupper T S. A Novel Method for the Isolation of Skin Resident T Cells from Normal and Diseased Human Skin. J Invest Dermatol. 2006; 126(5):1059-70.
21. Purwar R, Campbell J, Murphy G, Richards W G, Clark R A, Kupper T S. Resident memory T cells (T(RM)) are abundant in human lung: diversity, function, and antigen specificity. PLoS One. 2011; 6(1):e16245. doi: 10.1371/journal.pone.0016245. PubMed PMID: 21298112; PubMed Central PMCID: PMC3027667.
22. Trimble C L, Clark R A, Thoburn C, Hanson N C, Tassello J, Frosina D, Kos F, Teague J, Jiang Y, Barat N C, Jungbluth A A. Human Papillomavirus 16-Associated Cervical Intraepithelial Neoplasia in Humans Excludes CD8 T Cells from Dysplastic Epithelium. The Journal of Immunology. 2010; 185(11):7107-14. doi: 10.4049/jimmunol.1002756.
23. Booth J S, Toapanta F R, Salerno-Goncalves R, Patil S, Kader H, Safta A, Czinn S, Greenwald B, Sztein M B. Characterization and functional properties of gastric tissue-resident memory T cells from children, adults and the elderly. Frontiers in Immunology. 2014;5. doi: 10.3389/fimmu.2014.00294.
24. Liyanage U K, Moore T T, Joo H-G, Tanaka Y, Herrmann V, Doherty G, Drebin J A, Strasberg S M, Eberlein T J, Goedegebuure P S, Linehan D C. Prevalence of Regulatory T Cells Is Increased in Peripheral Blood and Tumor Microenvironment of Patients with Pancreas or Breast Adenocarcinoma. The Journal of Immunology. 2002; 169(5):2756-61. doi: 10.4049/jimmunol.169.5.2756.
25. Sieling P A, Torrelles J B, Stenger S, Chung W, Burdick A E, Rea T H, Brennan P J, Belisle J T, Porcelli S A, Modlin R L. The Human CD1-Restricted T Cell Repertoire Is Limited to Cross-Reactive Antigens: Implications for Host Responses against Immunologically Related Pathogens. The Journal of Immunology. 2005; 174(5):2637-44.
26. Krutzik S R, Tan B, Li H, Ochoa M T, Liu P T, Sharfstein S E, Graeber T G, Sieling P A, Liu Y-J, Rea T H, Bloom B R, Modlin R L. TLR activation triggers the rapid differentiation of monocytes into macrophages and dendritic cells. Nat Med. 2005; 11(6):653-60.
27. Gattinoni L, Klebanoff C A, Restifo N P. Paths to stemness: building the ultimate antitumour T cell. Nat Rev Cancer. 2012; 12(10):671-84. doi: 10.1038/nrc3322. PubMed PMID: 22996603.
28. Nakamoto N, Cho H, Shaked A, Olthoff K, Valiga M E, Kaminski M, Gostick E, Price D A, Freeman G J, Wherry E J, Chang K-M. Synergistic Reversal of Intrahepatic HCV-Specific CD8 T Cell Exhaustion by Combined PD-1/CTLA-4 Blockade. PLoS Pathog. 2009; 5(2):e1000313. doi: 10.1371/journal.ppat.1000313.
29. Barber D L, Wherry E J, Masopust D, Zhu B, Allison J P, Sharpe A H, Freeman G J, Ahmed R. Restoring function in exhausted CD8 T cells during chronic viral infection. Nature. 2006; 439(7077):682-7. doi: http://www.nature.com/nature/journal/v439/n7077/suppinfo/nature04444_S1.html.
30. Jones R B, Ndhlovu L C, Barbour J D, Sheth P M, Jha A R, Long B R, Wong J C, Satkunarajah M, Schweneker M, Chapman J M, Gyenes G, Vali B, Hyrcza M D, Yue F Y, Kovacs C, Sassi A, Loutfy M, Halpenny R, Persad D, Spotts G, Hecht F M, Chun T-W, McCune J M, Kaul R, Rini J M, Nixon D F, Ostrowski M A. Tim-3 expression defines a novel population of dysfunctional T cells with highly elevated frequencies in progressive HIV-1 infection. The Journal of Experimental Medicine. 2008; 205(12):2763-79. doi: 10.1084/jem.20081398.
31. Sakuishi K, Apetoh L, Sullivan J M, Blazar B R, Kuchroo V K, Anderson A C. Targeting Tim-3 and PD-1 pathways to reverse T cell exhaustion and restore anti-tumor immunity. The Journal of Experimental Medicine. 2010; 207(10):2187-94. doi: 10.1084/jem.20100643.
32. Purwar R, Schlapbach C, Xiao S, Kang H S, Elyaman W, Jiang X, Jetten A M, Khoury S J, Fuhlbrigge R C, Kuchroo V K, Clark R A, Kupper T S. Robust tumor immunity to melanoma mediated by interleukin-9-producing T cells. Nat Med. 2012; 18(8):1248-53. doi:http://www.nature.com/nm/journal/v18/n8/abs/nm.2856. html#supplementary-information.
33. Clark R A, Watanabe R, Teague J E, Schlapbach C, Tawa M C, Adams N, Dorosario A A, Chaney K S, Cutler C S, LeBoeuf N R, Carter J B, Fisher D C, Kupper T S. Skin Effector Memory T Cells Do Not Recirculate and Provide Immune Protection in Alemtuzumab-Treated CTCL Patients. Science Translational Medicine. 2012; 4(117):117ra7. doi: 10.1126/scitranslmed.3003008.
34. Golub T R, Slonim D K, Tamayo P, Huard C, Gaasenbeek M, Mesirov J P, Cotler H, Loh M L, Downing J R, Caligiuri M A, Bloomfield C D, Lander E S. Molecular Classification of Cancer: Class Discovery and Class Prediction by Gene Expression Monitoring. Science. 1999; 286(5439):531-7. doi: 10.1126/science.286.5439.531.
35. Ochoa M T, Stenger S, Sieling P A, Thoma-Uszynski S, Sabet S, Cho S, Krensky A M, Rollinghoff M, Nunes Sarno E, Burdick A E, Rea T H, Modlin R L. T-cell release of granulysin contributes to host defense in leprosy. Nat Med. 2001; 7(2):174-9.
36. Krutzik S R, Ochoa M T, Sieling P A, Uematsu S, Ng Y W, Legaspi A, Liu P T, Cole S T, Godowski P J, Maeda Y, Sarno E N, Norgard M V, Brennan P J, Akira S, Rea T H, Modlin R L. Activation and regulation of Toll-like receptors 2 and 1 in human leprosy. Nat Med. 2003; 9(5):525-32.
37. Ruffell B, Au A, Rugo H S, Esserman L J, Hwang E S, Coussens L M. Leukocyte composition of human breast cancer. Proceedings of the National Academy of Sciences. 2012; 109(8):2796-801. doi: 10.1073/pnas.1104303108.
38. Sprent J, Zhang X, Sun S, Tough D. T-Cell turnover in vivo and the role of cytokines. Immunology Letters. 1999; 65(1-2):21-5. doi: http://dx.doi.org/10.1016/S0165-2478(98)00119-9.
39. Lodolce J P, Burkett P R, Boone D L, Chien M, Ma A. T Cell-Independent Interleukin 15ra Signals Are Required for Bystander Proliferation. The Journal of Experimental Medicine. 2001; 194(8):1187-94. doi: 10.1084/jem.194.8.1187.
40. Liu W, Putnam A L, Xu-yu Z, Szot G L, Lee M R, Zhu S, Gottlieb P A, Kapranov P, Gingeras T R, de St. Groth B F, Clayberger C, Soper D M, Ziegler S F, Bluestone J A. CD127 expression inversely correlates with FoxP3 and suppressive function of human CD4+ T reg cells. The Journal of Experimental Medicine. 2006; 203(7):1701-11. doi: 10.1084/jem.20060772.
41. Acosta-Rodriguez E V, Napolitani G, Lanzavecchia A, Sallusto F. Interleukins 1b and 6 but not transforming growth factor-b are essential for the differentiation of interleukin 17-producing human T helper cells. Nat Immunol. 2007; 8(9):942-9. doi: http://www.nature.com/ni/journal/v8/n9/suppinfo/ni1496_S1.html.
42. Monney L, Sabatos C A, Gaglia J L, Ryu A, Waldner H, Chernova T, Manning S, Greenfield E A, Coyle A J, Sobel R A, Freeman G J, Kuchroo V K. Th1-specific cell surface protein Tim-3 regulates macrophage activation and severity of an autoimmune disease. Nature. 2002; 415(6871):536-41. Epub 2002Jan. 2. doi: 10.1038/415536a 415536a [pii]. PubMed PMID: 11823861.
43. Ochoa M-T, Stenger S, Sieling P A, Thoma-Uszynski S, Sabet S, Cho S, Krensky A M, Rollinghoff M, Nunes Sarno E, Burdick A E, Rea T H, Modlin R L. T-cell release of granulysin contributes to host defense in leprosy. Nat Med. 2001; 7(2):174-9.

What is claimed is:

1. A method for predicting in a subject having metastatic breast cancer whether the metastatic breast cancer is stable or progressive, the method comprising:
measuring two or more T cell subsets in a fluid sample obtained from the subject;
calculating the ratio of the T cell subsets; and
predicting whether the metastatic breast cancer is stable or progressive, according to the ratio of the T cell subsets.

2. The method of claim 1, wherein the T cell subsets include regulatory T cells (Treg) and CD4 T cells.

3. The method of claim 2, wherein:
the ratio of the T cell subsets is Treg/CD4 T cells; and
the subject's disease is more likely to be progressive metastatic cancer if the ratio of the T cell subsets is greater than about 0.30, greater than about 0.29, greater than about 0.28, greater than about 0.27, greater than about 0.26, greater than about 0.25, greater than about 0.24, greater than about 0.23, greater than about 0.22, or greater than about 0.21, greater than about 0.20, greater than about 0.19, greater than about 0.18, greater than about 0.17, greater than about 0.16, greater than about 0.15, greater than about 0.14, greater than about 0.13, greater than about 0.12, or greater than about 0.11.

4. The method of claim 1, wherein the fluid sample is peripheral blood.

5. A method for treating a subject having a stable metastatic breast cancer or a progressive metastatic breast cancer, the method comprising:
measuring two or more T cell subsets in a fluid sample obtained from the subject;
calculating the ratio of the T cell subsets;
predicting whether the metastatic breast cancer is stable or progressive, according to the ratio of the T cell subsets; and
administering a conservative therapy to the subject having a stable metastatic breast cancer, or administering an aggressive therapy to the subject having a progressive metastatic breast cancer.

6. The method of claim 5, wherein the ratio of the T cell subsets is Treg/CD4 T cells; and the ratio is greater than about 0.30, greater than about 0.29, greater than about 0.28, greater than about 0.27, greater than about 0.26, greater than about 0.25, greater than about 0.24, greater than about 0.23, greater than about 0.22, or greater than about 0.21, greater than about 0.20, greater than about 0.19, greater than about 0.18, greater than about 0.17, greater than about 0.16, greater than about 0.15, greater than about 0.14, greater than about 0.13, greater than about 0.12, or greater than about 0.11.

7. The method of claim 6, wherein the subject has a progressive metastatic breast cancer.

8. The method of claim 5, wherein the fluid sample is a blood sample.
